(19) United States
(12) Patent Application Publication
(10) Pub. No.: US 2003/0186925 A1
(43) Pub. Date: Oct. 2, 2003

(54) Farnesyl protein transferase inhibitor combinations with anti-tumor nucleoside derivatives

(76) Inventors: Peter Albert Palmer, Keerbergen (BE); Ivan David Horak, Yardley, PA (US)

Correspondence Address: JOE D. TAYLOR, 480 CONTENTMENT RD., MAKANDA, IL, 62958, US

(21) Appl. No.: 10/220,395

(22) PCT Filed: Feb. 26, 2001
(86) PCT No.: PCT/EP01/02164

(30) Foreign Application Priority Data
Feb. 29, 2000 (EP) 00200697.1

(51) Int. Cl. A61K 31/7076; A61K 31/7072; A61K 31/52; A61K 31/517; A61K 31/513; A61K 31/4745; A61K 31/4709

(52) U.S. Cl. 514/46; 514/263.23; 514/266.31; 514/291; 514/312; 514/314; 514/269; 514/49

(57) ABSTRACT

The present invention is concerned with combinations of a farnesyl transferase inhibitor and an anti-tumor nucleoside derivative for inhibiting the growth of tumor cells and useful in the treatment of cancer.

The present invention is concerned with combinations of a farnesyl transferase inhibitor and an anti-tumor nucleoside derivative for inhibiting the growth of tumor cells. and useful in the treatment of cancer.

Oncogenes frequently encode protein components of signal transduction pathways which lead to stimulation of cell growth and mitogenesis. Oncogene expression in cultured cells leads to cellular transformation, characterized by the ability of cells to grow in soft agar and the growth of cells as dense foci lacking the contact inhibition exhibited by non-transformed cells. Mutation and/or overexpression of certain oncogenes is frequently associated with human cancer. A particular group of oncogenes is known as ras which have been identified in mammals, birds, insects, mollusks, plants, fungi and yeasts. The family of mammalian ras oncogenes consists of three major members (“isoforms”): H-ras, K-ras and N-ras oncogenes. These ras oncogenes code for highly related proteins generically known as p21^{ras}. Once attached to plasma membranes, the mutant or oncogenic forms of p21^{ras} will provide a signal for the transformation and uncontrolled growth of malignant tumor cells. To acquire this transforming potential, the precursor of the p21^{ras} oncoprotein must undergo an enzymatically catalyzed farnesylation of the cysteine residue located in a carboxyl-terminal tetrapeptide. Therefore, inhibitors of the enzyme that catalyzes this modification, farnesyl protein transferase, will prevent the membrane attachment of p21^{ras} and block the aberrant growth of ras-transformed tumors. Hence, it is generally accepted in the art that farnesyl transferase inhibitors can be very useful as anticancer agents for tumors in which ras contributes to transformation.

Since mutated, oncogenic forms of ras are frequently found in many human cancers, most notably in more than 50% of colon and pancreatic carcinomas (Kohl et al., Science, vol 260, 1834-1837, 1993), it has been suggested that farnesyl tranferase inhibitors can be very useful against these types of cancer. Following further investigations, it has been found that a farnesyl transferase inhibitor is capable of demonstrating antiproliferative effects in vitro and anti-tumor effects in vivo in a variety of human tumor cell lines with and without ras gene mutations.

WO-97/21701 describes the preparation, formulation and pharmaceutical properties of farnesyl protein transferase inhibiting (imidazoly-5-yl)methyl-2-quinolinone derivatives of formulas (I), (II) and (III), as well as intermediates of formula (II) and (III) that are metabolized in vivo to the compounds of formula (I). The compounds of formulas (I). (II) and (III) are represented by

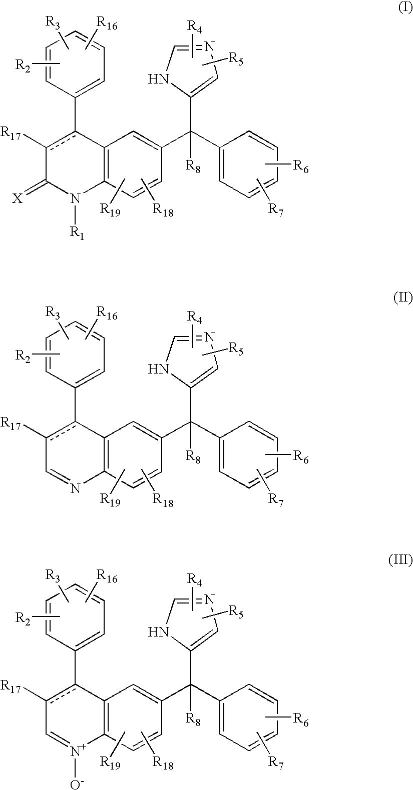

the pharmaceutically acceptable acid or base addition salts and the stereochemically isomeric forms thereof, wherein

the dotted line represents an optional bond;

X is oxygen or sulfur;

R¹ is hydrogen, C₁₋₁₂alkyl, Ar¹, Ar²C₁₋₆alkyl, quinolinylC₁₋₆alkyl, pyridylC₁₋₆alkyl, hydroxyC₁₋₆alkyl, C₁₋₆alkyloxyC₁₋₆alkyl, mono- or di(C₁₋₆alkyl)aminoC₁₋₆alkyl, aminoC₁₋₆alkyl,

or a radical of formula -Alk¹—C(═O)—R⁹, -Alk¹—S(O)—R⁹ or -Alk¹-S(O)₂—R⁹,

wherein Alk¹ is C₁₋₆alkanediyl,

R⁹ is hydroxy, C₁₋₆alkyl, C₁₋₆alkyloxy, amino, C₁₋₈alkylamino or C₁₋₈alkylamnino substituted with C₁₋₆alkyloxycarbonyl;

R², R³ and R¹⁶ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, aminoC₁₋₆alkyloxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl, 4,4-dimethyloxazolyl; or

when on adjacent positions R² and R³ taken together may form a bivalent radical of formula

| | |
|---|---|
| —O—CH₂—O— | (a-1), |
| —O—CH₂—CH₂—O— | (a-2), |
| —O—CH═CH— | (a-3), |
| —O—CH₂—CH₂— | (a-4), |
| —O—CH₂—CH₂—CH₂— | (a-5), or |
| —CH═CH—CH═CH— | (a-6); |

R⁴ and R⁵ each independently are hydrogen, halo, Ar¹, C₁₋₆alkyl, hydroxyC₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;

R⁶ and R⁷ each independently are hydrogen, halo, cyano, C₁₋₆alkyl, C₁₋₆alkyloxy, Ar²oxy, trihalomethyl, C₁₋₆alkylthio, di(C₁₋₆alkyl)amino, or when on adjacent positions R⁶ and R⁷ taken together may form a bivalent radical of formula

| | |
|---|---|
| —O—CH₂—O— | (c-1), or |
| —CH═CH—CH═CH— | (c-2); |

R⁸ is hydrogen, C₁₋₆alkyl, cyano, hydroxycarbonyl, C₁₋₆alkyloxycarbonyl, C₁₋₆alkylcarbonylC₁₋₆alkyl, cyanoC₁₋₆alkyl, C₁₋₆alkyloxycarbonylC₁₋₆alkyl, carboxyC₁₋₆alkyl, hydroxyC₁₋₆alkyl, aminoC₁₋₆alkyl, mono- or di(C₁₋₆alkyl)-aminoC₁₋₆alkyl, imidazolyl, haloC₁₋₆alkyl, C₁₋₆alkyloxyC₁₋₆alkyl, aminocarbonylC₁₋₆alkyl, or a radical of formula

| | |
|---|---|
| —O—R¹⁰ | (b-1), |
| —S—R¹⁰ | (b-2), |
| —N—R¹¹R¹² | (b-3), |

wherein R¹⁰ is hydrogen, C₁₋₆alkyl, C₁₋₆alkylcarbonyl, Ar¹, Ar²C₁₋₆alkyl, C₁₋₆alkyloxycarbonylC₁₋₆alkyl, or a radical or formula -Alk²-OR¹³ or -Alk²-NR¹⁴R¹⁵;

R¹¹ is hydrogen, C₁₋₁₂alkyl, Ar¹ or Ar²C₁₋₆alkyl;

R¹² is hydrogen, C₁₋₆alkyl, C₁₋₁₆alkylcarbonyl, C₁₋₆alkyloxycarbonyl, C₁₋₆alkylaminocarbonyl, Ar¹, Ar²C₁₋₆alkyl, C₁₋₆alkylcarbonyl-C₁₋₆alkyl, a natural amino acid, Ar¹carbonyl, Ar²C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino, or a radical or formula -Alk²-OR¹³ or -Alk²-NR¹⁴R¹⁵;

wherein Alk² is C₁₋₆alkanediyl;

R¹³ is hydrogen, C₁₋₆alkyl, C₁₋₆alkylcarbonyl, hydroxyC₁₋₆alkyl, Ar¹ or Ar²C₁₋₆alkyl;

R¹⁴ is hydrogen, C₁₋₆alkyl, Ar¹ or Ar²C₁₋₆alkyl;

R¹⁵ is hydrogen, C₁₋₆alkyl, C₁₋₆alkylcarbonyl, Ar¹ or Ar²C₁₋₆alkyl;

R¹⁷ is hydrogen, halo, cyano, C₁₋₆alkyl, C₁₋₆alkyloxycarbonyl, Ar¹;

R¹⁸ is hydrogen, C₁₋₆alkyl, C₁₋₆alkyloxy or halo;

R¹⁹ is hydrogen or C₁₋₆alkyl;

Ar¹ is phenyl or phenyl substituted with C₁₋₆alkyl, hydroxy, amino, C₁₋₆alkyloxy or halo; and

Ar² is phenyl or phenyl substituted with C₁₋₆alkyl, hydroxy, amino, C₁₋₆alkyloxy or halo.

WO-97/16443 concerns the preparation, formulation and pharmaceutical properties of farnesyl protein transferase inhibiting compounds of formula (IV), as well as intermediates of formula (V) and (VI) that are metabolized in vivo to the compounds of formula (IV). The compounds of formulas (IV), (V) and (VI) are represented by

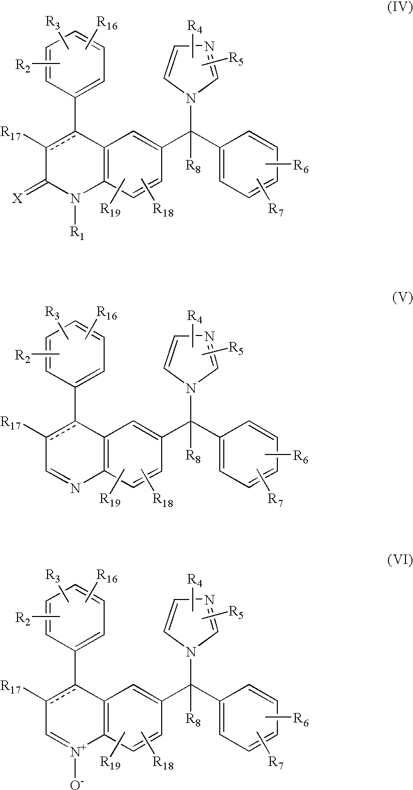

the pharmaceutically acceptable acid or base addition salts and the stereochemically isomeric forms thereof, wherein

the dotted line represents an optional bond;

X is oxygen or sulfur;

R¹ is hydrogen, C₁₋₁₂alkyl, Ar¹, Ar²C₁₋₆alkyl, quinolinylC₁₋₆alkyl, pyridylC₁₋₆alkyl, hydroxyC₁₋₆alkyl, C₁₋₆alkyloxyC₁₋₆alkyl, mono- or di(C₁₋₆alkyl)aminoC₁₋₆alkyl, aminoC₁₋₆alkyl,

or a radical of formula -Alk¹-C(═O)—R⁹, -Alk¹-S(O)—R⁹ or —Alk¹-S(O)₂—R⁹,

wherein Alk¹ is C₁₋₆alkanediyl,

R⁹ is hydroxy, C₁₋₆alkyl, C₁₋₆alkyloxy, amino, C₁₋₈alkylamino or C₁₋₈alkylamino substituted with C₁₋₆alkyloxycarbonyl;

R² and R³ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, amino-C₁₋₆alkyloxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl; or

when on adjacent positions R² and R³ taken together may form a bivalent radical of formula

| | |
|---|---|
| —O—CH₂—O— | (a-1), |
| —O—CH₂—CH₂—O— | (a-2), |
| —O—CH═CH— | (a-3), |
| —O—CH₂—CH₂— | (a-4), |
| —O—CH₂—CH₂—CH₂— | (a-5), or |
| —CH═CH—CH═CH— | (a-6); |

R⁴ and R⁵ each independently are hydrogen, Ar¹, C₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;

R⁶ and R⁷ each independently are hydrogen, halo, cyano, C₁₋₆alkyl, C₁₋₆alkyloxy or Ar²oxy;

R⁸ is hydrogen, C₁₋₆alkyl, cyano, hydroxycarbonyl, C₁₋₆alkyloxycarbonyl, C₁₋₆alkylcarbonylC₁₋₆alkyl, cyanoC₁₋₆alkyl, C₁₋₆alkyloxycarbonylC₁₋₆alkyl, hydroxycarbonylC₁₋₆alkyl, hydroxyC₁₋₆alkyl, aminoC₁₋₆alkyl, mono- or di(C₁₋₆alkyl)aminoC₁₋₆alkyl, haloC₁₋₆alkyl, C₁₋₆alkyloxyC₁₋₆alkyl, aminocarbonylC₁₋₆alkyl, Ar¹, Ar²C₁₋₆alkyloxyC₁₋₆alkyl, C₁₋₆alkylthioC₁₋₆alkyl;

R¹⁰ is hydrogen, C₁₋₆alkyl, C₁₋₆alkyloxy or halo;

R¹¹ is hydrogen or C₁₋₆alkyl;

Ar¹ is phenyl or phenyl substituted with C₁₋₆alkyl,hydroxy, amino, C₁₋₆alkyloxy or halo;

Ar² is phenyl or phenyl substituted with C₁₋₆alkyl,hydroxy, amino, C₁₋₆alkyloxy or halo.

WO-98/40383 concerns the preparation, formulation and pharmaceutical properties of farnesyl protein transferase inhibiting compounds of formula (VII)

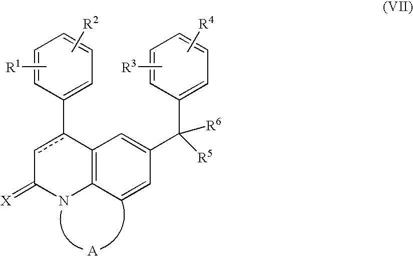

the pharmaceutically acceptable acid addition salts and the stereochemically isomeric forms thereof, wherein

the dotted line represents an optional bond;

X is oxygen or sulfur;

—A— is a bivalent radical of formula

| | | | |
|---|---|---|---|
| —CH═CH— | (a-1), | —CH₂—S— | (a-6), |
| —CH₂—CH₂— | (a-2), | —CH₂—CH₂—S— | (a-7), |
| —CH₂—CH₂—CH₂— | (a-3), | —CH═N— | (a-8), |
| —CH₂—O— | (a-4), | —N═N— | (a-9), or |
| —CH₂—CH₂—O— | (a-5), | —CO—NH— | (a-10); |

wherein optionally one hydrogen atom may be replaced by C₁₋₄alkyl or Ar¹;

R¹ and R² each independently are hydrogen, hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkyloxyC₁₋₆alkyloxy, C₁₋₆alkyloxycarbonyl, aminoC₁₋₆alkyloxy, mono- or di(C₁₋₆alkyl)aminoC₁₋₆alkyloxy, Ar², Ar²-C₁₋₆alkyl, Ar²-oxy, Ar²-C₁₋₆alkyloxy; or when on adjacent positions R¹ and R² taken together may form a bivalent radical of formula

| | |
|---|---|
| —O—CH₂—O— | (b-1), |
| —O—CH₂—CH₂—O— | (b-2), |
| —O—CH═CH— | (b-3), |
| —O—CH₂—CH₂— | (b-4), |
| —O—CH₂—CH₂—CH₂— | (b-5), or |
| —CH═CH—CH═CH— | (b-6); |

R³ and R⁴ each independently are hydrogen, halo, cyano, C₁₋₆alkyl, C₁₋₆alkyloxy, Ar³-oxy, C₁₋₆alkylthio, di(C₁₋₆alkyl)amino, trihalomethyl, trihalomethoxy, or when on adjacent positions R³ and R⁴ taken together may form a bivalent radical of formula

| | |
|---|---|
| —O—CH₂—O— | (c-1), |
| —O—CH₂—CH₂—O— | (c-2), or |
| —CH═CH—CH═CH— | (c-3); |

R⁵ is a radical of formula

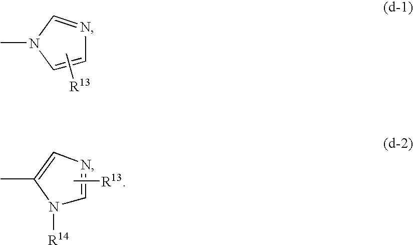

wherein R¹³ is hydrogen, halo, Ar⁴, C₁₋₆alkyl, hydroxyC₁₋₆alkyl, C₁₋₆alkyloxyC₁₋₆alkyl, C₁₋₆alkyloxy, C₁₋₆alkylthio, amino, C₁₋₆alkyloxycarbonyl, C₁₋₆alkylS(O)C₁₋₆alkyl or C₁₋₆alkylS(O)₂C₁₋₆alkyl;

R¹⁴ is hydrogen, C₁₋₆alkyl or di(C₁₋₄alkyl)aminosulfonyl;

R⁶ is hydrogen, hydroxy, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, C₁₋₆alkyloxycarbonylC₁₋₆alkyl, C₁₋₆alkylcarbonyl-C₁₋₆alkyl, C₁₋₆alkyloxycarbonyl, mono- or di(C₁₋₆alkyl)aminoC₁₋₆alkyl, Ar⁵, Ar⁵-C₁₋₆alkyloxyC₁₋₆alkyl; or a radical of formula

—O—R⁷ (e-1),

—S—R⁷ (e-2),

—N—R⁸R⁹ (e-3),

wherein R⁷ is hydrogen, C₁₋₆alkyl, C₁₋₆alkylcarbonyl, Ar⁶, Ar⁶—C₁₋₆alkyl, C₁₋₆alkyloxycarbonylC₁₋₆alkyl, or a radical of formula -Alk-OR¹⁰ or -Alk-NR¹¹R¹²;

R⁸ is hydrogen, C₁₋₆alkyl, Ar⁷ or Ar⁷-C₁₋₆alkyl;

R⁹ is hydrogen, C₁₋₆alkyl, C₁₋₆alkylcarbonyl, C₁₋₆alkyloxycarbonyl, C₁₋₆alkylaminocarbonyl, Ar⁸, Ar⁸—C₁₋₆alkyl, C₁₋₆alkylcarbonyl-C₁₋₆alkyl, Ar⁸-carbonyl, Ar⁸—C₁₋₆alkylcarbonyl, aminocarbonyl-carbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino, or a radical or formula -Alk-OR¹⁰ or -Alk-NR¹¹R¹²;

wherein Alk is C₁₋₆alkanediyl;

R¹⁰ is hydrogen, C₁₋₆alkyl, C₁₋₆alkylcarbonyl, hydroxyC₁₋₆alkyl, Ar⁹ or Ar⁹—C₁₋₆alkyl;

R¹¹ is hydrogen, C₁₋₆alkyl, C₁₋₆alkylcarbonyl, Ar¹⁰ or Ar¹⁰—C₁₋₆alkyl;

R¹² is hydrogen, C₁₋₆alkyl, Ar¹¹ or Ar¹¹—C₁₋₆alkyl; and

Ar¹ to Ar¹¹ are each independently selected from phenyl; or phenyl substituted with halo, C₁₋₆alkyl, C₁₋₆alkyloxy or trifluoromethyl.

WO-98/49157 concerns the preparation, formulation and pharmaceutical properties of farnesyl protein transferase inhibiting compounds of formula (VIII)

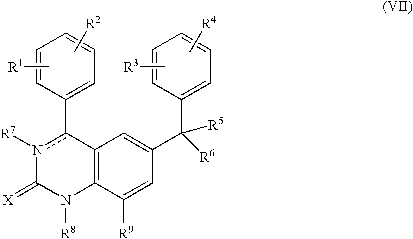

the pharmaceutically acceptable acid addition salts and the stereochemically isomeric forms thereof, wherein

the dotted line represents an optional bond;

X is oxygen or sulfur;

R¹ and R² each independently are hydrogen, hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkyloxyC₁₋₆alkyloxy, C₁₋₆alkyloxycarbonyl, aminoC₁₋₆alkyloxy, mono- or di(C₁₋₆alkyl)aminoC₁₋₆alkyloxy, Ar¹, Ar¹C₁₋₆alkyl, Ar¹oxy or Ar¹C₁₋₆alkyloxy;

R³ and R⁴ each independently are hydrogen, halo, cyano, C₁₋₆alkyl, C₁₋₆alkyloxy, Ar¹oxy, C₁₋₆alkylthio, di(C₁₋₆alkyl)amino, trihalomethyl or trihalomethoxy;

R⁵ is hydrogen, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, C₁₋₆alkyloxycarbonylC₁₋₆alkyl, C₁₋₆alkylcarbonyl-C₁₋₆alkyl, C₁₋₆alkyloxycarbonyl, mono- or di(C₁₋₆alkyl)aminoC₁₋₆alkyl, Ar¹, Ar¹C₁₋₆alkyloxyC₁₋₆alkyl; or a radical of formula

| | |
|---|---|
| —O—R¹⁰ | (a-1), |
| —S—R¹⁰ | (a-2), |
| —N—R¹¹R¹² | (a-3), |

wherein R¹⁰ is hydrogen, C₁₋₆alkyl, C₁₋₆alkylcarbonyl, Ar¹, Ar¹C₁₋₆alkyl, C₁₋₆alkyloxycarbonylC₁₋₆alkyl, or a radical of formula -Alk-OR¹³ or -Alk-NR¹⁴R¹⁵;

R¹¹ is hydrogen, C₁₋₆alkyl, Ar¹ or Ar¹C₁₋₆alkyl;

R¹² is hydrogen, C₁₋₆alkyl, C₁₋₆alkylcarbonyl, C₁₋₆alkyloxycarbonyl, C₁₋₆alkylaminocarbonyl, Ar¹, Ar¹C₁₋₆alkyl, C₁₋₆alkylcarbonyl-C₁₋₆alkyl, Ar¹carbonyl, Ar¹C₁₋₆alkylcarbonyl, aminocarbonyl-carbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino, or a radical or formula -Alk-OR¹³ or -Alk-NR¹⁴R¹⁵; wherein Alk is C₁₋₆alkanediyl;

R¹³ is hydrogen, C₁₋₆alkyl, C₁₋₆alkylcarbonyl, hydroxyC₁₋₆alkyl, Ar¹ or Ar¹C₁₋₆alkyl;

R¹⁴ is hydrogen, C₁₋₆alkyl, Ar¹ or Ar¹C₁₋₆alkyl;

R¹⁵ is hydrogen, C₁₋₆alkyl, C₁₋₆alkylcarbonyl, Ar¹ or Ar¹C₁₋₆alkyl;

R⁶ is a radical of formula

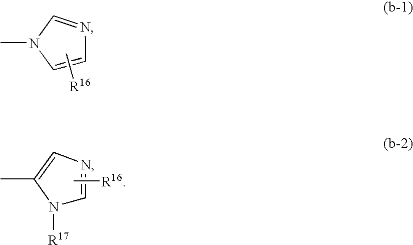

wherein R¹⁶is hydrogen, halo, Ar¹, C₁₋₆alkyl, hydroxyC₁₋₆alkyl, C₁₋₆alkyloxyC₁₋₆alkyl, C₁₋₆alkyloxy, C₁₋₆alkylthio, amino, C₁₋₆alkyloxycarbonyl, C₁₋₆alkylthioC₁₋₆alkyl, C₁₋₆alkylS(O)C₁₋₆alkyl or C₁₋₆alkylS(O)₂C₁₋₆alkyl;

R¹⁷ is hydrogen, C₁₋₆alkyl or di(C₁₋₄alkyl)aminosulfonyl;

R⁷ is hydrogen or C₁₋₆alkyl provided that the dotted line does not represent a bond;

R⁸ is hydrogen, C₁₋₆alkyl or Ar²CH₂ or Het¹CH₂;

R⁹ is hydrogen, C₁₋₆alkyl, C₁₋₆alkyloxy or halo; or

R⁸ and R⁹ taken together to form a bivalent radical of formula

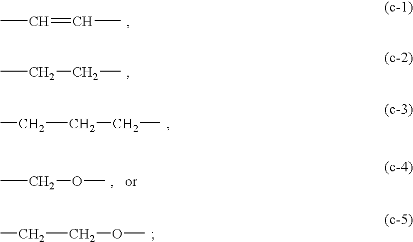

Ar¹ is phenyl; or phenyl substituted with 1 or 2 substituents each independently selected from halo, C₁₋₆alkyl, C₁₋₆alkyloxy or trifluoromethyl;

Ar² is phenyl; or phenyl substituted with 1 or 2 substituents each independently selected from halo, C₁₋₆alkyl, C₁₋₆alkyloxy or trifluoromethyl; and

Het¹ is pyridinyl; pyridinyl substituted with 1 or 2 substituents each independently selected from halo, C₁₋₆alkyl, C₁₋₆alkyloxy or trifluoromethyl.

WO-00/39082 concerns the preparation, formulation and pharmaceutical properties of farnesyl protein transferase inhibiting compounds of formula (IX)

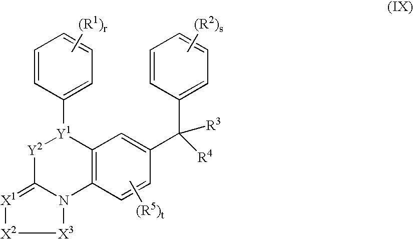

or the pharmaceutically acceptable acid addition salts and the stereochemically isomeric forms thereof, wherein

═X¹—X²—X³— is a trivalent radical of formula

| | | | |
|---|---|---|---|
| ═N—CR⁶═CR⁷— | (x-1), | ═CR⁶—CR⁷═CR⁸— | (x-6), |
| ═N—N═CR⁶— | (x-2), | ═CR⁶—N═CR⁷— | (x-7), |
| ═N—NH—C(═O)— | (x-3), | ═CR⁶—NH—C(═O)— | (x-8), or |
| ═N—N═N— | (x-4), | ═CR⁶—N═N— | (x-9); |
| ═N—CR⁶═N— | (x-5), | | |

wherein each R⁶, R⁷ and R⁸ are independently hydrogen, C₁₋₄alkyl, hydroxy, C₁₋₄alkyloxy, aryloxy, C₁₋₄alkyloxycarbonyl, hydroxyC₁₋₄alkyl, C₁₋₄alkyloxyC₁₋₄alkyl, mono- or di(C₁₋₄alkyl)aminoC₁₋₄alkyl, cyano, amino, thio, C₁₋₄alkylthio, arylthio or aryl;

>Y¹—Y²— is a trivalent radical of formula

| | |
|---|---|
| >CH—CHR⁹— | (y-1), |
| >C═N— | (y-2), |
| >CH—NR⁹— | (y-3), or |
| >C═CR⁹— | (y-4); |

wherein each R⁹ independently is hydrogen, halo, halocarbonyl, aminocarbonyl, hydroxyC₁₋₄alkyl, cyano, carboxyl, C₁₋₄alkyl, C₁₋₄alkyloxy, C₁₋₄alkyloxyC₁₋₄alkyl, C₁₋₄alkyloxycarbonyl, mono- or di(C₁₋₄alkyl)amino, mono- or di(C₁₋₄alkyl)aminoC₁₋₄alkyl, aryl;

r and s are each independently 0, 1, 2, 3, 4 or 5;

t is 0, 1, 2 or 3;

each R¹ and R² are independently hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkylthio, C₁₋₆alkyloxyC₁₋₆alkyloxy, C₁₋₆alkyloxycarbonyl, aminoC₁₋₆alkyloxy, mono- or di (C₁₋₆alkyl)amino, mono- or di(C₁₋₆alkyl)aminoC₁₋₆alkyloxy, aryl, arylC₁₋₆alkyl, aryloxy or arylC₁₋₆alkyloxy, hydroxycarbonyl, C₁₋₆alkyloxycarbonyl, aminocarbonyl, aminoC₁₋₆alkyl, mono- or di(C₁₋₆alkyl)aminocarbonyl, mono- or di(C₁₋₆alkyl)aminoC₁₋₆alkyl; or

two R¹ or R² substituents adjacent to one another on the phenyl ring may independently form together a bivalent radical of formula

| | |
|---|---|
| —O—CH₂—O— | (a-1), |
| —O—CH₂—CH₂—O— | (a-2), |
| —O═CH═CH— | (a-3), |
| —O—CH₂—CH₂— | (a-4), |
| —O—CH₂—CH₂—CH₂— | (a-5), or |
| —CH═CH—CH═CH— | (a-6); |

R³ is hydrogen, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, hydroxycarbonyl, hydroxycarbonylC₁₋₆alkyl, C₁₋₆alkyloxycarbonylC₁₋₆alkyl, C₁₋₆alkylcarbonylC₁₋₆alkyl, C₁₋₆alkyloxycarbonyl, aryl, arylC₁₋₆alkyloxyC₁₋₆alkyl, mono- or di(C₁₋₆alkyl)aminoC₁₋₆alkyl;

or a radical of formula

| | |
|---|---|
| —O—R¹⁰ | (b-1), |
| —S—R¹⁰ | (b-2), |
| —NR¹¹R¹² | (b-3), |

wherein R¹⁰ is hydrogen, C₁₋₆alkyl, C₁₋₆alkylcarbonyl, aryl, arylC₁₋₆alkyl, C₁₋₆alkyloxycarbonylC₁₋₆alkyl, or a radical of formula -Alk-OR¹³ or -Alk-NR¹⁴R¹⁵;

R¹¹ is hydrogen, C₁₋₆alkyl, aryl or arylC₁₋₆alkyl;

R¹² is hydrogen, C₁₋₆alkyl, aryl, hydroxy, amino, C₁₋₆alkyloxy, C₁₋₆alkylcarbonylC₁₋₆alkyl, arylC₁₋₆alkyl, C₁₋₆alkylcarbonylamino, mono- or di(C₁₋₆alkyl)amino, C₁₋₆alkylcarbonyl, aminocarbonyl, arylcarbonyl, haloC₁₋₆alkylcarbonyl, arylC₁₋₆alkylcarbonyl, C₁₋₆alkyloxycarbonyl, C₁₋₆alkyloxyC₁₋₆alkylcarbonyl, mono- or di(C₁₋₆alkyl)aminocarbonyl wherein the alkyl moiety may optionally be substituted by one or more substituents independently selected from aryl or C₁₋₃alkyloxycarbonyl, aminocarbonylcarbonyl, mono- or di(C₁₋₆alkyl)aminoC₁₋₆alkylcarbonyl, or a radical or formula -Alk-OR¹³ or -Alk-NR¹⁴R¹⁵;

wherein Alk is C₁₋₆alkanediyl;

R¹³ is hydrogen, C₁₋₆alkyl, C₁₋₆alkylcarbonyl, hydroxyC₁₋₆alkyl, aryl or arylC₁₋₆alkyl;

R¹⁴ is hydrogen, C₁₋₆alkyl, aryl or arylC₁₋₆alkyl;

R¹⁵ is hydrogen, C₁₋₆alkyl, C₁₋₆alkylcarbonyl, aryl or arylC₁₋₆alkyl;

R⁴ is a radical of formula

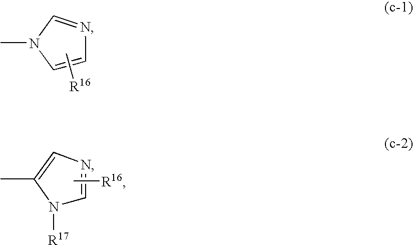

wherein R¹⁶ is hydrogen, halo, aryl, C₁₋₆alkyl, hydroxyC₁₋₆alkyl, C₁₋₆alkyloxyC₁₋₆alkyl, C₁₋₆alkyloxy, C₁₋₆alkylthio, amino, mono- or di(C₁₋₄alkyl)amino, hydroxycarbonyl, C₁₋₆alkyloxycarbonyl, C₁₋₆alkylthioC₁₋₆alkyl, C₁₋₆alkylS(O)C₁₋₆alkyl or C₁₋₆alkylS(O)₂C₁₋₆alkyl;

R¹⁶ may also be bound to one of the nitrogen atoms in the imidazole ring of formula (c-1) or (c-2), in which case the meaning of R¹⁶ when bound to the nitrogen is limited to hydrogen, aryl, C₁₋₆alkyl, hydroxyC₁₋₆alkyl, C₁₋₆alkyloxyC₁₋₆alkyl, C₁₋₆alkyloxycarbonyl, C₁₋₆alkylS(O)C₁₋₆alkyl or C₁₋₆alkylS(O)₂C₁₋₆alkyl;

R¹⁷ is hydrogen, C₁₋₆alkyl, C₁₋₆alkyloxyC₁₋₆alkyl, arylC₁₋₆alkyl, trifluoromethyl or di(C₁₋₄alkyl)aminosulfonyl;

R⁵ is C₁₋₆alkyl, C₁₋₆alkyloxy or halo;

aryl is phenyl, naphthalenyl or phenyl substituted with 1 or more substituents each independently selected from halo, C₁₋₆alkyl, C₁₋₆alkyloxy or trifluoromethyl.

Anti-tumor nucleoside derivatives have been used for many years for the treatment of various cancers. Among the oldest and most widely used of these derivatives is 5-fluorouracil (5-FU) which has been been used to treat a number of cancers such as colorectal, breast, hepatic and head and neck tumors. In order to enhance the cytotoxic effect of 5-FU, leucovorin (5-formyltetrahydrofolate) has been used with the drug to modulate levels of thymidylate synthase which are critical to ensure that malignant cells are sensitive to the effect of 5-FU. However, various factors limit the use of 5-FU, for example tumor resistance, toxicities, including gastrointestinal and haematological effects, and the need for intravenous administration. Various approaches have been taken to overcome these disadvantages including proposals to overcome the poor bioavailability of 5-FU and also to increase the therapeutic index of 5-FU, either by reducing systemic toxicity or by increasing the amount of active drug reaching the tumor. One such compound which provides improved therapeutic advantage over 5-FU is capecitabine, which has the chemical name [1-(5-deoxy-beta-D-ribofuranosyl)-5-fluoro-1,2-dihydro-2-oxopyrimidin-4-yl]-carbamic acid, pentyl ester. Capecitabine is a pro-drug of 5-FU which is well absorbed after oral dosing and delivers pharmacologically-active concentrations of 5-FU to tumors, with little systemic exposure to the active drug. As well as offering potentially superior activity to 5-FU, it can also be used for oral therapy with prolonged administration. Another anti-tumor nucleoside derivative is gemcitabine which has the chemical name 2′-deoxy-2′,2′-difluoro-cytidine, and which has been used in the treatment of various cancers including non-small cell lung cancer and pancreatic cancer.

Although anti-tumor nucleoside derivatives have widely used as chemotherapeutic agents in humans, they are not therapeutically effective in all patients or against all types of tumors.

There is therefore a need to increase the inhibitory efficacy of anti-tumor nucleoside derivatives against tumor growth and also to provide a means for the use of lower dosages of anti-tumor nucleoside derivatives to reduce the potential of adverse toxic side effects to the patient.

It is an object of the invention to provide a therapeutic combination of an anti-tumor nucleoside derivative and a farnesyl transferase inhibitor of the type described above which has an advantageous inhibitory effect against tumor cell growth, in comparison with the respective effects shown by the individual components of the combination.

According to the invention therefore we provide a combination of an anti-tumor nucleoside derivative and a farnesyl transferase inhibitor of formula (I), (II), (III), (IV), (V), (VI), (VII), (VIII) or (IX) above, in particular a compound of formula (I), (II) or (III):

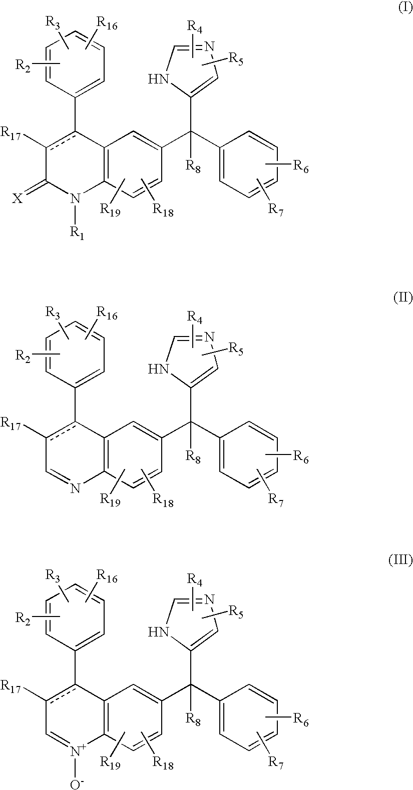

the pharmaceutically acceptable acid or base addition salts and the stereochemically isomeric forms thereof, wherein

the dotted line represents an optional bond;

X is oxygen or sulfur;

R¹ is hydrogen, C₁₋₁₂alkyl, Ar¹, Ar²C₁₋₆alkyl, quinolinylC₁₋₆alkyl, pyridylC₁₋₆alkyl, hydroxyC₁₋₆alkyl, C₁₋₆alkyloxyC₁₋₆alkyl, mono- or di(C₁₋₆alkyl)aminoC₁₋₆alkyl, aminoC₁₋₆akyl,

or a radical of formula -Alk¹-C(═O)—R⁹, -Alk¹-S(O)—R⁹ or -Alk¹-S(O)₂—R⁹,

wherein Alk¹ is C₁₋₆alkanediyl,

R⁹ is hydroxy, C₁₋₆alkyl, C₁₋₆alkyloxy, amino, C₁₋₈alkylamino or C₁₋₈alkylamino substituted with C₁₋₆alkyloxycarbonyl;

R², R³ and R¹⁶ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, aminoC₁₋₆alkyloxy, mono- or di(C₁₋₆alkyl)aminoC₁₋₆alkyloxy, Ar¹, Ar²C₁₋₆alkyl, Ar²oxy, Ar²C₁₋₆akyloxy, hydroxycarbonyl, C₁₋₆alkyloxycarbonyl, trihalomethyl, trihalomethoxy, C₂₋₆alkenyl, 4,4-dimethyloxazolyl; or

when on adjacent positions R² and R³ taken together may form a bivalent radical of formula

| | |
|---|---|
| —O—CH₂—O— | (a-1), |
| —O—CH₂—CH₂—O— | (a-2), |
| —O—CH═CH— | (a-3), |
| —O—CH₂—CH₂— | (a-4), |
| —O—CH₂—CH₂—CH₂— | (a-5), or |
| —CH═CH—CH═CH— | (a-6); |

R⁴ and R⁵ each independently are hydrogen, halo, Ar¹, C₁₋₆alkyl, hydroxyC₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;

R⁶ and R⁷ each independently are hydrogen, halo, cyano, C₁₋₆alkyl, C₁₋₆alkyloxy, Ar²oxy, trihalomethyl, C₁₋₆alkylthio, di(C₁₋₆alkyl)amino, or when on adjacent positions R⁶ and R⁷ taken together may form a bivalent radical of formula

| | |
|---|---|
| —O—CH₂—O— | (c-1), or |
| —CH═CH—CH═CH— | (c-2); |

R⁸ is hydrogen, C₁₋₆alkyl, cyano, hydroxycarbonyl, C₁₋₆alkyloxycarbonyl, C₁₋₆alkylcarbonylC₁₋₆alkyl, cyanoC₁₋₆alkyl, C₁₋₆alkyloxycarbonylC₁₋₆alkyl, carboxyC₁₋₆alkyl, hydroxyC₁₋₆alkyl, aminoC₁₋₆alkyl, mono- or di(C₁₋₆alkyl)aminoC₁₋₆alkyl, imidazolyl, haloC₁₋₆alkyl, C₁₋₆alkyloxyC₁₋₆alkyl, aminocarbonylC₁₋₆alkyl, or a radical of formula

| | |
|---|---|
| —O—R¹⁰ | (b-1), |
| —S—R¹⁰ | (b-2), |
| —N—R¹¹R¹² | (b-3), |

wherein R¹⁰ is hydrogen, C₁₋₆alkyl, C₁₋₆alkylcarbonyl, Ar¹, Ar²C₁₋₆alkyl, C₁₋₆alkyloxycarbonylC₁₋₆alkyl, or a radical or formula -Alk²-OR¹³ or -Alk²-NR¹⁴R¹⁵;

R¹¹ is hydrogen, C₁₋₁₂alkyl, Ar¹ or Ar²C₁₋₆alkyl;

R¹² is hydrogen, C₁₋6alkyl, C₁₋₁₆alkylcarbonyl, C₁₋₆alkyloxycarbonyl, C₁₋₆alkylaminocarbonyl, Ar¹, Ar²C₁₋₆alkyl, C₁₋₆alkylcarbonylC₁₋₆alkyl, a natural amino acid, Ar¹carbonyl, Ar²C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino, or a radical or formula -Alk²-OR¹³ or -Alk²-NR¹⁴R¹⁵;

wherein Alk² is C₁₋₆alkanediyl;

R¹³ is hydrogen, C₁₋₆alkyl, C₁₋₆alkylcarbonyl, hydroxyC₁₋₆alkyl, Ar¹ or Ar²C₁₋₆alkyl;

R¹⁴ is hydrogen, C₁₋₆alkyl, Ar¹ or Ar²C₁₋₆alkyl;

R¹⁵ is hydrogen, C₁₋₆alkyl, C₁₋₆alkylcarbonyl, Ar¹ or Ar²C₁₋₆alkyl;

R¹⁷ is hydrogen, halo, cyano, C₁₋₆alkyl, C₁₋₆alkyloxycarbonyl, Ar¹;

R¹⁸is hydrogen, C₁₋₆alkyl, C₁₋₆alkyloxy or halo;

R¹⁹ is hydrogen or C₁₋₆alkyl;

Ar¹ is phenyl or phenyl substituted with C₁₋₆alkyl, hydroxy, amino, C₁₋₆alkyloxy or halo; and

Ar² is phenyl or phenyl substituted with C₁₋₆alkyl, hydroxy, amino, C₁₋₆alkyloxy or halo.

The above described combinations are hereinafter referred to as combinations according to the invention. These combinations may provide a synergistic effect whereby they demonstrate an advantageous therapeutic effect which is greater than that which would have been expected from the effects of the individual components of the combinations.

In Formulas (I), (II) and (III), R⁴ or R⁵ may also be bound to one of the nitrogen atoms in the imidazole ring. In that case the hydrogen on the nitrogen is replaced by R⁴ or R⁵ and the meaning of R⁴ and R⁵ when bound to the nitrogen is limited to hydrogen, Ar¹, C₁₋₆alkyl, hydroxyC₁₋₆alkyl, C₁₋₆alkyloxyC₁₋₆alkyl, C₁₋₆alkyloxycarbonyl, C₁₋₆alkylS(O)C₁₋₆alkyl, C₁₋₆alkylS(O)₂C₁₋₆alkyl.

Preferably the substituent R¹⁸ is situated on the 5 or 7 position of the quinolinone moiety and substituent R¹⁹ is situated on the 8 position when R¹⁸ is on the 7-position.

Interesting compounds are these compounds of formula (I) wherein X is oxygen.

Also interesting compounds are these compounds of formula (I) wherein the dotted line represents a bond, so as to form a double bond.

Another group of interesting compounds are those compounds of formula (I) wherein R¹ is hydrogen, C₁₋₆alkyl, C₁₋₆alkyloxyC₁₋₆alkyl, di(C₁₋₆alkyl)aminoC₁₋₆alkyl, or a radical of formula -Alk¹-C(═O)—R⁹, wherein Alk¹ is methylene and R⁹ is C₁₋₈alkyl-amino substituted with C₁₋₆alkyloxycarbonyl.

Still another group of interesting compounds are those compounds of formula (I) wherein R³ is hydrogen or halo; and R² is halo, C₁₋₆alkyl, C₂₋₆alkenyl, C₁₋₆alkyloxy, trihalomethoxy or hydroxyC₁₋₆alkyloxy.

A further group of interesting compounds are those compounds of formula (I) wherein R² and R³ are on adjacent positions and taken together to form a bivalent radical of formula (a-1), (a-2) or (a-3).

A still further group of interesting compounds are those compounds of formula (I) wherein R⁵ is hydrogen and R⁴ is hydrogen or C₁₋₆alkyl.

Yet another group of interesting compounds are those compounds of formula (I) wherein R⁷ is hydrogen; and R⁶ is C₁₋₆alkyl or halo, preferably chloro, especially 4-chloro.

A particular group of compounds are those compounds of formula (I) wherein R⁸ is hydrogen, hydroxy, haloC₁₋₆alkyl, hydroxyC₁₋₆alkyl, cyanoC₁₋₆alkyl, C₁₋₆alkyloxycarbonylC₁₋₆alkyl, imidazolyl, or a radical of formula —NR¹¹R¹² wherein R¹¹ is hydrogen or C₁₋₁₂alkyl and R¹² is hydrogen, C₁₋₆alkyl, C₁₋₆alkyloxy, hydroxy, C₁₋₆alkyloxyC₁₋₆alkylcarbonyl, or a radical of formula -Alk²-OR¹³ wherein R¹³ is hydrogen or C₁₋₆alkyl.

Preferred compounds are those compounds wherein R¹ is hydrogen, C₁₋₆alkyl, C₁₋₆alkyloxyC₁₋₆alkyl, di(C₁₋₆alkyl)aminoC₁₋₆alkyl, or a radical of formula -Alk¹-C(═O)—R⁹, wherein Alk¹ is methylene and R⁹ is C₁₋₈alkylamino substituted with C₁₋₆alkyloxycarbonyl; R² is halo, C₁₋₆alkyl, C₂₋₆alkenyl, C₁₋₆alkyloxy, trihalomethoxy, hydroxyC₁₋₆alkyloxy or Ar¹; R³ is hydrogen; R⁴ is methyl bound to the nitrogen in 3-position of the imidazole; R⁵ is hydrogen; R⁶ is chloro; R⁷ is hydrogen; R⁸ is hydrogen, hydroxy, haloC₁₋₆alkyl, hydroxyC₁₋₆alkyl, cyanoC₁₋₆alkyl, C₁₋₆alkyloxycarbonylC₁₋₆alkyl, imidazolyl, or a radical of formula —NR¹¹R¹² wherein R¹¹ is hydrogen or C₁₋₁₂alkyl and R¹² is hydrogen, C₁₋₆alkyl, C₁₋₆alkyloxy, C₁₋₆alkyloxyC₁₋₆alkylcarbonyl, or a radical of formula -Alk²-OR¹³ wherein R¹³ is C₁₋₆alkyl; R¹⁷ is hydrogen and R¹⁸ is hydrogen.

Most preferred compounds are

4-(3-chlorophenyl)-6-[(4-chlorophenyl)hydroxy(1-methyl-1H-imidazol-5-yl)methyl]-1-methyl-2(1H)-quinolinone,

6-[amino(4-chlorophenyl)-1-methyl-1H-imidazol-5-ylmethyl]-4-(3-chlorophenyl)-1-methyl-2(1H)-quinolinone;

6-[(4-chlorophenyl)hydroxy(1-methyl-1H-imidazol-5-yl)methyl]-4-(3-ethoxyphenyl)-1-methyl-2(1H)-quinolinone;

6-[(4-chlorophenyl)(1-methyl-1H-imidazol-5-yl)methyl]-4-(3-ethoxyphenyl)-1-methyl-2(1H)-quinolinone monohydrochloride.monohydrate;

6-[amino(4-chlorophenyl)(1-methyl-1H-imidazol-5-yl)methyl]-4-(3-ethoxyphenyl)-1-methyl-2(1H)-quinolinone,

6-amino(4-chlorophenyl)(1-methyl-1H-imidazol-5-yl)methyl]-1-methyl-4-(3-propylphenyl)-2(1H)-quinolinone; a stereoisomeric form thereof or a pharmaceutically acceptable acid or base addition salt; and

(+)-6-[amino(4-chlorophenyl)(1-methyl-1H-imidazol-5-yl)methyl]-4-(3-chlorophenyl)-1-methyl-2(1H)-quinolinone (Compound 75 in Table 1 of the Experimental part of WO-97/21701); or a pharmaceutically acceptable acid addition salt thereof. The latter compound is especially preferred.

Further preferred embodiments of the present invention include compounds of formula (IX) wherein one or more of the following restrictions apply:

═X¹—X²—X³ is a trivalent radical of formula (x-1), (x-2), (x-3), (x-4) or (x-9) wherein each R⁶ independently is hydrogen, C₁₋₄alkyl, C₁₋₄alkyloxycarbonyl, amino or aryl and R⁷ is hydrogen;

>y¹—y²— is a trivalent radical of formula (y-1), (y-2), (y-3), or (y-4) wherein each R⁹ independently is hydrogen, halo, carboxyl, C₁₋₄alkyl or C₁₋₄alkyloxycarbonyl;

r is 0, 1 or 2;

s is 0 or 1;

t is 0;

R¹ is halo, C₁₋₆alkyl or two R¹ substituents ortho to one another on the phenyl ring may independently form together a bivalent radical of formula (a-1);

R² is halo;

R³ is halo or a radical of formula (b-1) or (b-3) wherein

R¹⁰ is hydrogen or a radical of formula -Alk-OR¹³.

R¹¹ is hydrogen;

R¹² is hydrogen, C₁₋₆alkyl, C₁₋₆alkylcarbonyl, hydroxy, C₁₋₆alkyloxy or mono- or

di(C₁₋₆alkyl)aminoC₁₋₆alkylcarbonyl;

Alk is C₁₋₆alkanediyl and R¹³ is hydrogen;

R⁴ is a radical of formula (c-1) or (c-2) wherein

R¹⁶ is hydrogen, halo or mono- or di(C₁₋₄alkyl)amino;

R¹⁷ is hydrogen or C₁₋₆alkyl;

aryl is phenyl.

A particular group of compounds consists of those compounds of formula (IX) wherein ═X¹—X²—X³ is a trivalent radical of formula (x-1), (x-2), (x-3), (x4) or (x-9), >Y1-Y2 is a trivalent radical of formula (y-2), (y-3) or (y-4), r is 0 or 1, s is 1, t is 0, R¹ is halo, C₍₁₋₄₎alkyl or forms a bivalent radical of formula (a-1), R² is halo or C₁₋₄alkyl, R³ is hydrogen or a radical of formula (b-1) or (b-3), R⁴ is a radical of formula (c-1) or (c-2), R⁶ is hydrogen, C₁₋₄alkyl or phenyl, R⁷ is hydrogen, R⁹ is hydrogen or C₁₋₄alkyl, R¹⁰ is hydrogen or -Alk-OR¹³, R¹¹ is hydrogen and R¹² is hydrogen or C₁₋₆alkylcarbonyl and R¹³ is hydrogen;

Preferred compounds are those compounds of formula (IX) wherein ═X¹—X²—X³ is a trivalent radical of formula (x-1) or (x-4), >Y1-Y2 is a trivalent radical of formula (y-4), r is 0 or 1, s is 1, t is 0, R¹ is halo, preferably chloro and most preferably 3-chloro, R² is halo, preferably 4-chloro or 4-fluoro, R³ is hydrogen or a radical of formula (b-1) or (b-3), R⁴ is a radical of formula (c-1) or (c-2), R⁶ is hydrogen, R⁷ is hydrogen, R⁹ is hydrogen, R¹⁰ is hydrogen, R¹¹ is hydrogen and R¹² is hydrogen;

Other preferred compounds are those compounds of formula (IX) wherein ═X¹—X²—X³ is a trivalent radical of formula (x-2), (x-3) or (x-4), >Y1-Y2 is a trivalent radical of formula (y-2), (y-3) or (y-4), r and s are 1, t is 0, R¹ is halo, preferably chloro, and most preferably 3-chloro or R¹ is C₁₋₄alkyl, preferably 3-methyl, R² is halo, preferably chloro, and most preferably 4-chloro, R³ is a radical of formula (b-1) or (b-3), R⁴ is a radical of formula (c-2), R⁶ is C₁₋₄alkyl, R⁹ is hydrogen, R¹⁰ and R¹¹ are hydrogen and R¹² is hydrogen or hydroxy.

The most preferred compounds of formula (IX) are

7-[(4-fluorophenyl)(1H-imidazol-1-yl)methyl]-5-phenylimidazo[1,2-a]quinoline;

α-(4-chlorophenyl)-α-(1-methyl-1H-imidazol-5-yl)-5-phenylimidazo[1,2-a]quinoline-7-methanol;

5-(3-chlorophenyl)-α-(4-chlorophenyl)-α-(1-methyl-1H-imidazol-5-yl)-imidazo-[1,2-a]quinoline-7-methanol;

5-(3-chlorophenyl)-α-(4-chlorophenyl)-α-(1-methyl-1H-imidazol-5-yl)imidazo-[1,2-a]quinoline-7-methanamine;

5-(3-chlorophenyl)-α-(4-chlorophenyl)-α-(1-methyl-1H-imidazol-5-yl)tetrazolo-[1,5-a]quinoline-7-methanamine;

5-(3-chlorophenyl)-α-(4-chlorophenyl)-1-methyl-α-(1-methyl-1H-imidazol-5-yl)-1,2,4-triazolo[4,3-a]quinoline-7-methanol;

5-(3-chlorophenyl)-α-(4-chlorophenyl)-α-(1-methyl-1H-imidazol-5-yl)tetrazolo-[1,5-a]quinoline-7-methanamine;

5-(3-chlorophenyl)-α-(4-chlorophenyl)-α-(1-methyl-1H-imidazol-5-yl)tetrazolo-[1,5-a]quinazoline-7-methanol;

5-(3-chlorophenyl)-α-(4-chlorophenyl)-4,5-dihydro-α-(1-methyl-1H-imidazol-5-yl)-tetrazolo[1,5-a]quinazoline-7-methanol;

5-(3-chlorophenyl)-α-(4-chlorophenyl)-α-(1-methyl-1H-imidazol-5-yl)tetrazolo-[1,5-a]quinazoline-7-methanamine;

5-(3-chlorophenyl)-α-(4-chlorophenyl)-N-hydroxy-α-(1-methyl-1H-imidazol-5-yl)-tetrahydro[1,5-a]quinoline-7-methanamine;

α-(4-chlorophenyl)-α-(1-methyl-1H-imidazol-5-yl)-5-(3-methylphenyl)tetrazolo-[1,5-a]quinoline-7-methanamine; the pharmaceutically acceptable acid addition salts and the stereochemically isomeric forms thereof.

5-(3-chlorophenyl)-α-(4-chlorophenyl)-α-(1-methyl-1H-imidazol-5-yl)tetrazolo-[1,5-a]quinazoline-7-methanamine, especially the (−) enantiomer, and its pharmaceutically acceptable acid addition salts are especially preferred.

As used in the foregoing definitions and hereinafter halo defines fluoro, chloro, bromo and iodo; C₁₋₆alkyl defines straight and branched chained saturated hydrocarbon radicals having from 1 to 6 carbon atoms such as, for example, methyl, ethyl, propyl, butyl, pentyl, hexyl and the like; C₁₋₈alkyl encompasses the straight and branched chained saturated hydrocarbon radicals as defined in C₁₋₆alkyl as well as the higher homologues thereof containing 7 or 8 carbon atoms such as, for example heptyl or octyl; C₁₋₁₂alkyl again encompasses C₁₋₈alkyl and the higher homologues thereof containing 9 to 12 carbon atoms, such as, for example, nonyl, decyl, undecyl, dodecyl; C₁₋₁₆alkyl again encompasses C₁₋₁₂alkyl and the higher homologues thereof containing 13 to 16 carbon atoms, such as, for example, tridecyl, tetradecyl, pentedecyl and hexadecyl; C₂₋₆alkenyl defines straight and branched chain hydrocarbon radicals containing one double bond and having from 2 to 6 carbon atoms such as, for example, ethenyl, 2-propenyl, 3-butenyl, 2-pentenyl, 3-pentenyl, 3-methyl-2-butenyl, and the like; C₁₋₆alkanediyl defines bivalent straight and branched chained saturated hydrocarbon radicals having from 1 to 6 carbon atoms, such as, for example, methylene, 1,2-ethanediyl, 1,3-propanediyl, 1,4-butanediyl, 1,5-pentanediyl, 1,6-hexanediyl and the branched isomers thereof. The term “C(═O)” refers to a carbonyl group, “S(O)” refers to a sulfoxide and “S(O)₂” to a sulfon. The term “natural amino acid” refers to a natural amino acid that is bound via a covalent amide linkage formed by loss of a molecule of water between the carboxyl group of the amino acid and the amino group of the remainder of the molecule. Examples of natural amino acids are glycine, alanine, valine, leucine, isoleucine, methionine, proline, phenylanaline, tryptophan, serine, threonine, cysteine, tyrosine, asparagine, glutamine, aspartic acid, glutamic acid, lysine, arginine, histidine.

The pharmaceutically acceptable acid or base addition salts as mentioned hereinabove are meant to comprise the therapeutically active non-toxic acid and non-toxic base addition salt forms which the compounds of formulas (I), (II), (III), (IV), (V), (VI), (VII), (VIII) or (IX) are able to form. The compounds of formulas (I), (II), (III), (IV), (V), (VI), (VII), (VIII) or (IX) which have basic properties can be converted in their pharmaceutically acceptable acid addition salts by treating said base form with an appropriate acid. Appropriate acids comprise, for example, inorganic acids such as hydrohalic acids, e.g. hydrochloric or hydrobromic acid; sulfuric; nitric; phosphoric and the like acids; or organic acids such as, for example, acetic, propanoic, hydroxyacetic, lactic, pyruvic, oxalic, malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

The compounds of formulae (I), (II), (III), (IV), (V), (VI), (VII), (VIII) or (IX) which have acidic properties may be converted in their pharmaceutically acceptable base addition salts by treating said acid form with a suitable organic or inorganic base. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The terms acid or base addition salt also comprise the hydrates and the solvent addition forms which the compounds of formulae (I), (II), (III), (IV), (V), (VI), (VII), (VIII) or (IX) are able to form. Examples of such forms are e.g. hydrates, alcoholates and the like.

The term stereochemically isomeric forms of compounds of formulae (I), (II), (III), (IV), (V), (VI), (VII), (VIII) or (IX), as used hereinbefore, defines all possible compounds made up of the same atoms bonded by the same sequence of bonds but having different three-dimensional structures which are not interchangeable, which the compounds of formulae (I), (II), (III), (IV), (V), (VI), (VII), (VIII) or (IX) may possess. Unless otherwise mentioned or indicated, the chemical designation of a compound encompasses the mixture of all possible stereochemically isomeric forms which said compound may possess. Said mixture may contain all diastereomers and/or enantiomers of the basic molecular structure of said compound. All stereochemically isomeric forms of the compounds of formulae (I), (II), (III), (IV), (V), (VI), (VII), (VIII) or (IX) both in pure form or in admixture with each other are intended to be embraced within the scope of the present invention.

Some of the compounds of formulae (I), (II), (III), (IV), (V), (VI), (VII), (VIII) or (IX) may also exist in their tautomeric forms. Such forms although not explicitly indicated in the above formula are intended to be included within the scope of the present invention.

Whenever used hereinafter, the term “compounds of formulae (I), (II), (III), (IV), (V), (VI), (VII), (VIII) or (IX)” is meant to include also the pharmaceutically acceptable acid or base addition salts and all stereoisomeric forms.

Preferred anti-tumor nucleoside derivatives for use in accordance with the invention include 5-fluorouracil, gemcitabine and capecitabine referred to above. 5- Fluorouracil is widely available commercially, and may be prepared for example as described in U.S. Pat. No. 2,802,005. Gemcitabine is commercially available for example from Eli Lilly under the trade name Gemzar and may be prepared for example as described in European patent specification No. 122707 or by processes analogous thereto. Capecitabine is commercially available for example from Hoffman-La Roche under under the trade name Xeloda and may be prepared for example as described in European patent specification No. 698611 or by processes analogous thereto. Other anti-tumor nucleoside derivatives may be prepared in conventional manner for example by processes analogous to those described above for capecitabine and gemcitabine.

The present invention also relates to combinations according to the invention for use in medical therapy for example for inhibiting the growth of tumor cells.

The present invention also relates to the use of combinations according to the invention for the preparation of a pharmaceutical composition for inhibiting the growth of tumor cells.

The present invention also relates to a method of inhibiting the growth of tumor cells in a human subject which comprises administering to the subject an effective amount of a combination according to the invention.

This invention further provides a method for inhibiting the abnormal growth of cells, including transformed cells, by administering an effective amount of a combination according to the invention. Abnormal growth of cells refers to cell growth independent of normal regulatory mechanisms (e.g. loss of contact inhibition). This includes the abnormal growth of: (1) tumor cells (tumors) expressing an activated ras oncogene; (2) tumor cells in which the ras protein is activated as a result of oncogenic mutation of another gene; (3) benign and malignant cells of other proliferative diseases in which aberrant ras activation occurs. Furthermore, it has been suggested in literature that ras oncogenes not only contribute to the growth of of tumors in vivo by a direct effect on tumor cell growth but also indirectly, i.e. by facilitating tumor-induced angiogenesis (Rak. J. et al, Cancer Research, 55, 4575-4580, 1995). Hence, pharmacologically targetting mutant ras oncogenes could conceivably suppress solid tumor growth in vivo, in part, by inhibiting tumor-induced angiogenesis.

This invention also provides a method for inhibiting tumor growth by administering an effective amount of a combination according to the present invention, to a subject, e.g. a mammal (and more particularly a human) in need of such treatment. In particular, this invention provides a method for inhibiting the growth of tumors expressing an activated ras oncogene by the administration of an effective amount of combination according to the present invention. Examples of tumors which may be inhibited include, but are not limited to, lung cancer (e.g. adenocarcinoma and including non-small cell lung cancer), pancreatic cancers (e.g. pancreatic carcinoma such as, for example exocrine pancreatic carcinoma), colon cancers (e.g. colorectal carcinomas, such as, for example, colon adenocarcinoma and colon adenoma), hematopoietic tumors of lymphoid lineage (e.g. acute lymphocytic leukemia, B-cell lymphoma, Burkitt's lymphoma), myeloid leukemias (for example, acute myelogenous leukemia (AML)), thyroid follicular cancer, myelodysplastic syndrome (MDS), tumors of mesenchymal origin (e.g. fibrosarcomas and rhabdomyosarcomas), melanomas, teratocarcinomas, neuroblastomas, gliomas, benign tumor of the skin (e.g. keratoacanthomas), breast carcinoma (e.g. advanced breast cancer), kidney caminoma, ovary carcinoma, bladder carcinoma and epidermal carcinoma.

This invention also provides a method for inhibiting proliferative diseases, both benign and malignant, wherein ras proteins are aberrantly activated as a result of oncogenic mutation in genes, i.e. the ras gene itself is not activated by mutation to an oncogenic mutation to an oncogenic form, with said inhibition being accomplished by the administration of an effective amount of a combination according to the invention, to a subject in need of such a treatment. For example, the benign proliferative disorder neurofibromatosis, or tumors in which ras is activated due to mutation or overexpression of tyrosine kinase oncogenes may be inhibited by the combinations according to the invention.

The anti-tumor nucleoside derivative and the farnesyl transferase inhibitor may be administered simultaneously (e.g. in separate or unitary compositions) or sequentially in either order. In the latter case, the two compounds will be administered within a period and in an amount and manner that is sufficient to ensure that an advantageous or synergistic effect is achieved. It will be appreciated that the preferred method and order of administration and the respective dosage amounts and regimes for each component of the combination will depend on the particular anti-tumor nucleoside derivative and farnesyl transferase inhibitor being administered, their route of administration, the particular tumor being treated and the particular host being treated. The optimum method and order of administration and the dosage amounts and regime can be readily determined by those skilled in the art using conventional methods and in view of the information set out herein.

The farnesyl transferase inhibitor is advantageously administered in an effective amount of from 0.0001 mg/kg to 100 mg/kg body weight, and in particular from 0.001 mg/kg to 10 mg/kg body weight. More particularly, for an adult patient, the dosage is conveniently in the range of 50 to 500 mg bid, advantageously 100 to 400 mg bid and particularly 300 mg bid.

The anti-tumor nucleoside derivative is advantageously administered in a dosage of 200 to 2500 mg per square meter (mg/m²) of body surface area, for example 700 to1500 mg/m2, particularly for 5-FU in a dosage of 200 to 500 mg/m², for gemcitabine in a dosage of about 800 to 1200 mg/m² and for capecitabine in about 1000 to 2500 mg/m² per course of treatment. These dosages may be administered for example once, twice or more per course of treatment, which may be repeated for example every 7, 14, 21 or 28 days.

It is especially preferred to administer the farnesyl tranferase inhibitor at a dosage of 100 or 200 mg bid for 7, 14, 21 or 28 days with a dosage of the anti-tumor nucleoside derivative in the ranges indicated above.

In view of their useful pharmacological properties, the components of the combinations according to the invention, i.e. the anti-tumor nucleoside derivative and the farnesyl transferase inhibitor may be formulated into various pharmaceutical forms for administration purposes. The components may formulated separately in individual pharmaceutical compositions or in a unitary pharmaceutical composition containing both components. Farnesyl protein transferase inhibitors can be prepared and formulated into pharmaceutical compositions by methods known in the art and in particular according to the methods described in the published patent specifications mentioned herein and incorporated by reference; for the compounds of formulae (I), (II) and (III) suitable examples can be found in WO-97/21701. Compounds of formulae (IV), (V), and (VI) can be prepared and formulated using methods described in WO 97/16443, compounds of formulae (VII) and (VIII) according to methods described in WO 98/40383 and WO 98/49157 and compounds of formula (IX) according to methods described in WO 00/39082 respectively.

The present invention therefore also relates to a pharmaceutical composition comprising an anti-tumor nucleoside derivative compound and a farnesyl tranferase inhibitor of formula (I) together with one or more pharmaceutical carriers. To prepare pharmaceutical compositions for use in accordance with the invention, an effective amount of a particular compound, in base or acid addition salt form, as the active ingredient is combined in intimate admixture with a pharmaceutically acceptable carrier, which carrier may take a wide variety of forms depending on the form of preparation desired for administration. These pharmaceutical compositions are desirably in unitary dosage form suitable, preferably, for administration orally, rectally, percutaneously, or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, to aid solubility for example, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wetting agent, optionally combined with suitable additives of any nature in minor proportions, which additives do not cause a significant deleterious effect to the skin. Said additives may facilitate the administration to the skin and/or may be helpful for preparing the desired compositions. These compositions may be administered in various ways, e.g., as a transdermal patch, as a spot-on, as an ointment.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

It may be appropriate to administer the required dose of each component of the combination as two, three, four or more sub-doses at appropriate intervals throughout the course of treatment Said sub-doses may be formulated as unit dosage forms, for example, in each case containing independently 0.01 to 500 mg, for example 0.1 to 200 mg and in particular 1 to 100 mg of each active ingredient per unit dosage form.

### Experimental Testing of Combinations for Inhibition of Tumor Growth

The combinations according to the invention may be tested for their efficacy in inhibiting tumor growth using conventional assays described in the literature for example the HTB177 lung carcinoma described by Liu M et al, Cancer Research, Vol. 58, No.21, 1 Nov. 1998, pages 4947-4956, and the anti-mitotic assay described by Moasser M et al, Proc. Natl. Acad. Sci. USA, Vol. 95, pages 1369-1374, February 1998. Other in vitro and in vivo models for determining ant-tumor effects of combinations and possible synergy of the combinations according to the invention are described in WO 98/54966 and WO 98/32114. Clinical models for determining the efficacy and possible synergism for combination therapy in the clinic are generally described in Cancer: Principles and Practice of Oncology, Fifth Edition, edited by Vincent T DeVita, Jr., Samuel Hellman, Steven A. Rosenberg, Lippincott-Raven, Philadelphia, 1997, especially Chapter 17, pages 342-346.

1. A combination of an anti-tumor nucleoside derivative and a farnesyl transferase inhibitor selected from compounds of formulae (I), (II), (III), (IV), (V), (VI), (VII), (VIII) and (IX) below: 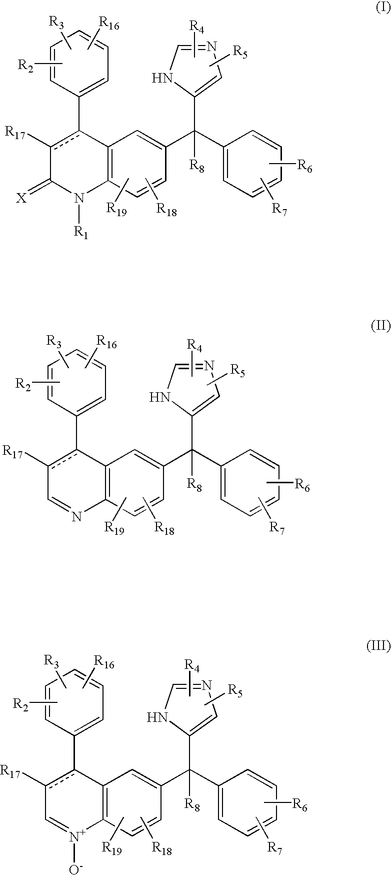
the pharmaceutically acceptable acid or base addition salts and the stereochemically isomeric forms thereof, wherein
the dotted line represents an optional bond;
X is oxygen or sulfur;
R¹ is hydrogen, C₁₋₁₂alkyl, Ar¹, Ar²C₁₋₆alkyl, quinolinylC₁₋₆alkyl, pyridylC₁₋₆alkyl, hydroxyC₁₋₆alkyl, C₁₋₆alkyloxyC₁₋₆alkyl, mono- or di(C₁₋₆alkyl)aminoC₁₋₆alkyl, aminoC₁₋₆alkyl,
or a radical of formula -Alk¹-C(═O)—R⁹, -Alk¹-S(O)—R⁹ or -Alk¹-S(O)₂—R⁹,
wherein Alk¹ is C₁₋₆alkanedlyl,
R⁹ is hydroxy, C₁₋₆alkyl, C₁₋₆alkyloxy, amino, C₁₋₈alkylamino or C₁₋₈alkylamino substituted with C₁₋₆alkyloxycarbonyl;
R², R³ and R¹⁶ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, aminoC₁₋₆alkyloxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl, 4,4-dimethyloxazolyl; or
when on adjacent positions R² and R³ taken together may form a bivalent radical of formula

| | |
|---|---|
| —O—CH₂—O— | (a-1), |
| —O—CH₂—CH₂—O— | (a-2), |
| —O—CH═CH— | (a-3), |
| —O—CH₂—CH₂— | (a-4), |
| —O—CH₂—CH₂—CH₂— | (a-5), or |
| —CH═CH—CH═CH— | (a-6); |

R⁴ and R⁵ each independently are hydrogen, halo, Ar¹, C₁₋₆alkyl, hydroxyC₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;
R⁶ and R⁷ each independently are hydrogen, halo, cyano, C₁₋₆alkyl, C₁₋₆alkyloxy, Ar²oxy, trihalomethyl, C₁₋₆alkylthio, di(C₁₋₆alkyl)amino, or when on adjacent positions R⁶ and R⁷ taken together may form a bivalent radical of formula

| | |
|---|---|
| —O—CH₂—O— | (c-1), or |
| —CH═CH—CH═CH— | (c-2); |

R⁸ is hydrogen, C₁₋₆alkyl, cyano, hydroxycarbonyl, C₁₋₆alkyloxycarbonyl, C₁₋₆alkylcarbonylC₁₋₆alkyl, cyanoC₁₋₆alkyl, C₁₋₆alkyloxycarbonylC₁₋₆alkyl, carboxyC₁₋₆alkyl, hydroxyC₁₋₆alkyl, aminoC₁₋₆alkyl, mono- or di(C₁₋₆alkyl)-aminoC₁₋₆alkyl, imidazolyl, haloC₁₋₆alkyl, C₁₋₆alkyloxyC₁₋₆alkyl, aminocarbonylC₁₋₆alkyl, or a radical of formula

| | |
|---|---|
| —O—R¹⁰ | (b-1), |
| —S—R¹⁰ | (b-2), |
| —N—R¹¹R¹² | (b-3), |

wherein R¹⁰ is hydrogen, C₁₋₆alkyl, C₁₋₆alkylcarbonyl, Ar¹, Ar²C₁₋₆alkyl, C₁₋₆alkyloxycarbonylC₁₋₆alkyl, or a radical or formula -Alk²-OR¹³ or -Alk²-NR¹⁴R¹⁵;
R¹¹ is hydrogen, C₁₋₁₂alkyl, Ar¹ or Ar²C₁₋₆alkyl;
R¹² is hydrogen, C₁₋₆alkyl, C₁₋₁₆alkylcarbonyl, C₁₋₆alkyloxycarbonyl, C₁₋₆alkylaminocarbonyl, Ar¹, Ar²C₁₋₆alkyl, C₁₋₆alkylcarbonyl-C₁₋₆alkyl, a natural amino acid, Ar¹carbonyl, Ar²C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino, or a radical or formula -Alk²-OR¹³ or -Alk²-NR¹⁴R¹⁵;
wherein Alk² is C₁₋₆alkanediyl;
R¹³ is hydrogen, C₁₋₆alkyl, C₁₋₆alkylcarbonyl, hydroxy-C₁₋₆alkyl, Ar¹ or Ar²C₁₋₆alkyl;
R¹⁴ is hydrogen, C₁₋₆alkyl, Ar¹ or Ar²C₁₋₆alkyl;
R¹⁵ is hydrogen, C₁₋₆alkyl, C₁₋₆alkylcarbonyl, Ar¹ or Ar²C₁₋₆alkyl;
R¹⁷ is hydrogen, halo, cyano, C₁₋₆alkyl, C₁₋₆alkyloxycarbonyl, Ar¹;
R¹⁸ is hydrogen, C₁₋₆alkyl, C₁₋₆alkyloxy or halo;
R¹⁹ is hydrogen or C₁₋₆alkyl;
Ar¹ is phenyl or phenyl substituted with C₁₋₆alkyl, hydroxy, amino, C₁₋₆alkyloxy or halo; and
Ar² is phenyl or phenyl substituted with C₁₋₆alkyl, hydroxy, amino, C₁₋₆alkyloxy or halo. 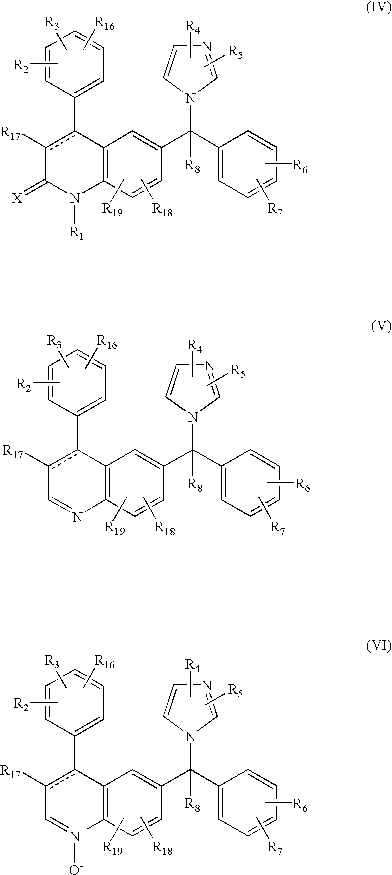
the pharmaceutically acceptable acid or base addition salts and the stereochemically isomeric forms thereof, wherein
the dotted line represents an optional bond;
X is oxygen or sulfur;
R¹ is hydrogen, C₁₋₁₂alkyl, Ar¹, Ar²C₁₋₆alkyl, quinolinylC₁₋₆alkyl, pyridyl-C₁₋₆alkyl, hydroxyC₁₋₆alkyl, C₁₋₆alkyloxyC₁₋₆alkyl, mono- or di(C₁₋₆alkyl)-aminoC₁₋₆alkyl, aminoC₁₋₆alkyl,
or a radical of formula -Alk¹-C(═O)—R⁹, -Alk¹-S(O)—R⁹ or -Alk¹-S(O)₂—R⁹,
wherein Alk¹ is C₁₋₆alkanediyl,
R⁹ is hydroxy, C₁₋₆alkyl, C₁₋₆alkyloxy, amino, C₁₋₈alkylamino or C₁₋₈alkylamino substituted with C₁₋₆alkyloxycarbonyl;
R² and R³ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, amino-C₁₋₆alkyloxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl; or
when on adjacent positions R² and R³ taken together may form a bivalent radical of formula

| | |
|---|---|
| —O—CH₂—O— | (a-1), |
| —O—CH₂—CH₂—O— | (a-2), |
| —O—CH═CH— | (a-3), |
| —O—CH₂—CH₂— | (a-4), |
| —O—CH₂—CH₂—CH₂— | (a-5), or |
| —CH═CH—CH═CH— | (a-6); |

R⁴ and R⁵ each independently are hydrogen, Ar¹, C₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;
R⁶ and R⁷ each independently are hydrogen, halo, cyano, C₁₋₆alkyl, C₁₋₆alkyloxy or Ar²oxy;
R⁸ is hydrogen, C₁₋₆alkyl, cyano, hydroxycarbonyl, C₁₋₆alkyloxycarbonyl, C₁₋₆alkyl-carbonylC₁₋₆alkyl, cyanoC₁₋₆alkyl, C₁₋₆alkyloxycarbonylC₁₋₆alkyl, hydroxy-carbonylC₁₋₆alkyl, hydroxyC₁₋₆alkyl, aminoC₁₋₆alkyl, mono- or di(C₁₋₆alkyl)-aminoC₁₋₆alkyl, haloC₁₋₆alkyl, C₁₋₆alkyloxyC₁₋₆alkyl, aminocarbonylC₁₋₆alkyl, Ar¹, Ar²C₁₋₆alkyloxyC₁₋₆alkyl, C₁₋₆alkylthioC₁₋₆alkyl;
R¹⁰ is hydrogen, C₁₋₆alkyl, C₁₋₆alkyloxy or halo;
R¹¹ is hydrogen or C₁₋₆alkyl;
Ar¹ is phenyl or phenyl substituted with C₁₋₆alkyl,hydroxy,amino,C₁₋₆alkyloxy or halo;
Ar² is phenyl or phenyl substituted with C₁₋₆alkyl,hydroxy, amino, C₁₋₆alkyloxy or halo. 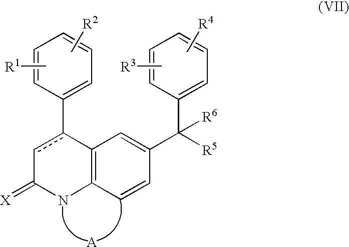
the pharmaceutically acceptable acid addition salts and the stereochemically isomeric forms thereof, wherein the dotted line represents an optional bond;
X is oxygen or sulfur;
—A— is a bivalent radical of formula

| | | | |
|---|---|---|---|
| —CH═CH— | (a-1), | —CH₂—S— | (a-6), |
| —CH₂—CH₂— | (a-2), | —CH₂—CH₂—S— | (a-7), |
| —CH₂—CH₂—CH₂— | (a-3), | —CH═N— | (a-8), |
| —CH₂—O— | (a-4), | —N═N— | (a-9), or |
| —CH₂—CH₂—O— | (a-5), | —CO—NH— | (a-10); |

wherein optionally one hydrogen atom may be replaced by C₁₋₄alkyl or Ar¹;
R¹ and R² each independently are hydrogen, hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkyloxyC₁₋₆alkyloxy, C₁₋₆alkyloxycarbonyl, aminoC₁₋₆alkyloxy, mono- or di(C₁₋₆alkyl)aminoC₁₋₆alkyloxy, Ar², Ar²—C₁₋₆alkyl, Ar²-oxy, Ar²—C₁₋₆alkyloxy; or when on adjacent positions R¹ and R² taken together may form a bivalent radical of formula

| | |
|---|---|
| —O—CH₂—O— | (b-1), |
| —O—CH₂—CH₂—O— | (b-2), |
| —O—CH═CH— | (b-3), |
| —O—CH₂—CH₂— | (b-4), |
| —O—CH₂—CH₂—CH₂— | (b-5), or |
| —CH═CH—CH═CH— | (b-6); |

R³ and R⁴ each independently are hydrogen, halo, cyano, C₁₋₆alkyl, C₁₋₆alkyloxy, Ar³-oxy, C₁₋₆alkylthio, di(C₁₋₆alkyl)amino, trihalomethyl, trihalomethoxy, or when on adjacent positions R³ and R⁴ taken together may form a bivalent radical of formula

| | |
|---|---|
| —O—CH₂—O— | (c-1), |
| —O—CH₂—CH₂—O— | (c-2), or |
| —CH═CH—CH═CH— | (c-3); |

R⁵ is a radical of formula 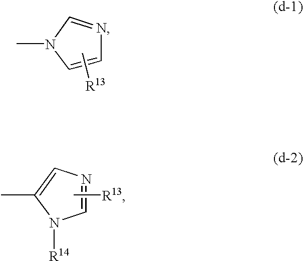
wherein R¹³ is hydrogen, halo, Ar⁴, C₁₋₆alkyl, hydroxyC₁₋₆alkyl, C₁₋₆alkyloxy-C₁₋₆alkyl, C₁₋₆alikyloxy, C₁₋₆alkylthio, amino, C₁₋₆alkyloxy-carbonyl, C₁₋₆alkylS(O)C₁₋₆alkyl or C₁₋₆alkylS(O)₂C₁₋₆alkyl;
R¹⁴is hydrogen, C₁₋₆alkyl or di(C₁₋₄alkyl)aminosulfonyl;
R⁶ is hydrogen, hydroxy, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, C₁₋₆alkyloxycarbonylC₁₋₆alkyl, C₁₋₆alkylcarbonyl-C₁₋₆alkyl, C₁₋₆alkyloxycarbonyl, mono- or di(C₁₋₆alkyl)aminoC₁₋₆alkyl, Ar⁵, Ar⁵—C₁₋₆alkyloxyC₁₋₆alkyl; or a radical of formula
—O—R⁷ (e-1),
—S—R⁷ (e-2),
—N—R⁸R⁹ (e-3),
wherein R⁷ is hydrogen, C₁₋₆alkyl, C₁₋₆alkylcarbonyl, Ar⁶, Ar⁶—C₁₋₆alkyl, C₁₋₆alkyloxycarbonylC₁₋₆alkyl, or a radical of formula -Alk-OR¹⁰ or -Alk-NR¹¹R¹²;
R⁸ is hydrogen, C₁₋₆alkyl, Ar⁷ or Ar⁷—C₁₋₆alkyl;
R⁹ is hydrogen, C₁₋₆alkyl, C₁₋₆alkylcarbonyl, C₁₋₆alkyloxycarbonyl, C₁₋₆alkylaminocarbonyl, Ar⁸, Ar⁸—C₁₋₆alkyl, C₁₋₆alkylcarbonyl-C₁₋₆alkyl, Ar⁸-carbonyl, Ar⁸—C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino, or a radical or formula -Alk-OR¹⁰ or -Alk-NR¹¹R¹²;
wherein Alk is C₁₋₆alkanediyl;
R¹⁰ is hydrogen, C₁₋₆alkyl, C₁₋₆alkylcarbonyl, hydroxyC₁₋₆alkyl, Ar⁹ or Ar⁹—C₁₋₆alkyl;
R¹¹ is hydrogen, C₁₋₆alkyl, C₁₋₆alkylcarbonyl, Ar¹⁰ or Ar¹⁰—C₁₋₆alkyl;
R¹² is hydrogen, C₁₋₆alkyl, Ar¹¹ or Ar¹¹—C₁₋₆alkyl; and
Ar¹ to Ar¹¹ are each independently selected from phenyl; or phenyl substituted with halo, C₁₋₆alkyl, C₁₋₆alkyloxy or trifluoromethyl. 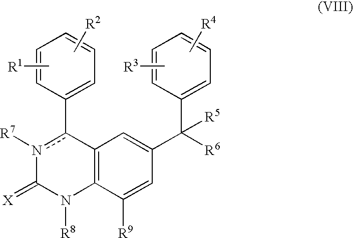
the pharmaceutically acceptable acid addition salts and the stereochermically isomeric forms thereof, wherein the dotted line represents an optional bond;
X is oxygen or sulfur;
R¹ and R² each independently are hydrogen, hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkyloxyC₁₋₆alkyloxy, C₁₋₆alkyloxyearbonyl, aminoC₁₋₆alkyloxy, mono- or di(C₁₋₆alkyl)aminoC₁₋₆alkyloxy, Ar¹, Ar¹C₁₋₆alkyl, Ar¹oxy or Ar¹C₁₋₆alkyloxy;
R³ and R⁴ each independently are hydrogen, halo, cyano, C₁₋₆alkyl, C₁₋₆alkyloxy, Ar¹oxy, C₁₋₆alkylthio, di(C₁₋₆alkyl)amino, trihalomethyl or trihalomethoxy;
R⁵ is hydrogen, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, C₁₋₆alkyloxycarbonylC₁₋₆alkyl, C₁₋₆alkylcarbonyl-C₁₋₆alkyl, C₁₋₆alkyloxycarbonyl, mono- or di(C₁₋₆alkyl)aminoC₁₋₆alkyl, Ar¹, Ar¹C₁₋₆alkyloxyC₁₋₆alkyl; or a radical of formula

| | |
|---|---|
| —O—R¹⁰ | (a-1), |
| —S—R¹⁰ | (a-2), |
| —N—R¹¹R¹² | (a-3), |

wherein R¹⁰ is hydrogen, C₁₋₆alkyl, C₁₋₆alkylcarbonyl, Ar¹, Ar¹C₁₋₆alkyl, C₁₋₆alkyloxycarbonylC₁₋₆alkyl, or a radical of formula -Alk-OR¹³ or -Alk-NR¹⁴R¹⁵;
R¹¹ is hydrogen, C₁₋₆alkyl, Ar¹ or Ar¹C₁₋₆alkyl;
R¹² is hydrogen, C₁₋₆alkyl, C₁₋₆alkylcarbonyl, C₁₋₆alkyloxyearbonyl, C₁₋₆alkylaminocarbonyl, Ar¹, Ar¹C₁₋₆alkyl, C₁₋₆alkylcarbonyl-C₁₋₆alkyl, Ar¹carbonyl, Ar¹C₁₋₆alkylcarbonyl, amninocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylearbonyl, amino, C₁₋₆alkylamino, C₁₋₆alkylcarbonylamino,
or a radical or formula -Alk-OR¹³ or -Alk-NR¹⁴R¹⁵;
wherein Alk is C₁₋₆alkanediyl;
R¹³ is hydrogen, C₁₋₆alkyl, C₁₋₆alkylcarbonyl, hydroxyC₁₋₆alkyl, Ar¹ or Ar¹C₁₋₆alkyl;
R¹⁴ is hydrogen, C₁₋₆alkyl, Ar¹ or Ar¹C₁₋₆alkyl;
R¹⁵ is hydrogen, C₁₋₆alkyl, C₁₋₆alkylcarbonyl, Ar¹ or Ar¹C₁₋₆alkyl;
R⁶ is a radical of formula 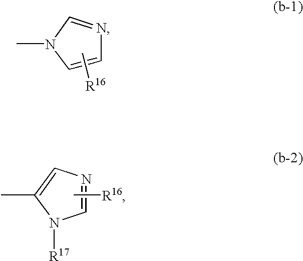
wherein R¹⁶is hydrogen, halo, Ar¹, C₁₋₆alkyl, hydroxyC₁₋₆alkyl, C₁₋₆alkyloxyC₁₋₆alkyl, C₁₋₆alkyloxy, C₁₋₆alkylthio, amino, C₁₋₆alkyloxycarbonyl, C₁₋₆alkylthioC₁₋₆alkyl, C₁₋₆alkylS(O)C₁₋₆alkyl or C₁₋₆alkylS(O)₂C₁₋₆alkyl;
R¹⁷ is hydrogen, C₁₋₆alkyl or di(C₁₋₄alkyl)aminosulfonyl;
R⁷ is hydrogen or C₁₋₆alkyl provided that the dotted line does not represent a bond;
R⁸ is hydrogen, C₁₋₆alkyl or Ar²CH₂ or Het¹CH₂;
R⁹ is hydrogen, C₁₋₆alkyl, C₁₋₆alkyloxy or halo; or
R⁸ and R⁹ taken together to form a bivalent radical of formula

| | |
|---|---|
| —CH═CH— | (c-1), |
| —CH₂—CH₂— | (c-2), |
| —CH₂—CH₂—CH₂— | (c-3), |
| —CH₂—O— | (c-4), or |
| —CH₂—CH₂—O— | (c-5); |

Ar¹ is phenyl; or phenyl substituted with 1 or 2 substituents each independently selected from halo, C₁₋₆alkyl, C₁₋₆alkyloxy or trifluoromethyl;
Ar² is phenyl; or phenyl substituted with 1 or 2 substituents each independently selected from halo, C₁₋₆alkyl, C₁₋₆alkyloxy or trifluoromethyl; and
Het¹ is pyridinyl; pyridinyl substituted with 1 or 2 substituents each independently selected from halo, C₁₋₆alkyl, C₁₋₆alkyloxy or trifluoromethyl
and 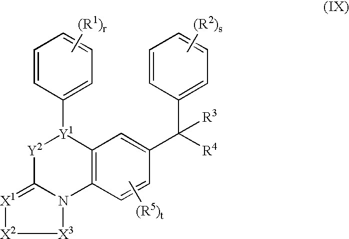
or the pharmaceutically acceptable acid addition salts and the stereochemrically isomeric forms thereof, wherein
═X¹—X²—X³— is a trivalent radical of formula

| | |
|---|---|
| ═N—CR⁶═CRhu 7— | (x-1), |
| ═N—N═CR⁶— | (x-2), |
| ═N—NH—C(═O)— | (x-3), |
| ═N—N═N— | (x-4), |
| ═N—CR⁶═N— | (x-5), |
| ═CR⁶—CR⁷═CR⁸— | (x-6), |
| ═CR⁶—N═CR⁷— | (x-7), |
| ═CR⁶—NH—C(═O)— | (x-8), or |
| ═CR⁶—N═N— | (x-9); |

wherein each R⁶, R⁷ and R⁸ are independently hydrogen, C₁₋₄alkyl, hydroxy, C₁₋₄alkyloxy, aryloxy, C₁₋₄alkyloxycarbonyl, hydroxyC₁₋₄alkyl, C₁₋₄alkyloxyC₁₋₄alkyl, mono- or di(C₁₋₄alkyl)aminoC₁₋₄alkyl, cyano, amino, thio, C₁₋₄alkylthio, arylthio or aryl;
>Y¹—Y²— is a trivalent radical of formula

| | |
|---|---|
| >CH—CHR⁹— | (y-1), |
| >C═N— | (y-2), |
| >CH—NR⁹— | (y-3),or |
| >C═CR⁹— | (y-4); |

wherein each R⁹ independently is hydrogen, halo, halocarbonyl, aminocarbonyl, hydroxyC₁₋₄alkyl, cyano, carboxyl, C₁₋₄alkyl, C₁₋₄alkyloxy, C₁₋₄alkyloxyC₁₋₄alkyl, C₁₋₄alkyloxycarbonyl, mono- or di(C₁₋₄alkyl)amino, mono- or di(C₁₋₄alkyl)aminoC₁₋₄alkyl, aryl;
r and s are each independently 0, 1, 2, 3, 4 or 5;
t is 0, 1, 2 or 3;
each R¹ and R² are independently hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkylthio, C₁₋₆alkyloxyC₁₋₆alkyloxy, C₁₋₆alkyloxycarbonyl, aminoC₁₋₆alkyloxy, mono- or di(C₁₋₆alkyl)amino, mono- or di(C₁₋₆alkyl)aminoC₁₋₆alkyloxy, aryl, arylC₁₋₆alkyl, aryloxy or arylC₁₋₆alkyloxy, hydroxycarbonyl, C₁₋₆alkyloxycarbonyl, aminocarbonyl, aminoC₁₋₆alkyl, mono- or di(C₁₋₆alkyl)aminocarbonyl, mono- or di(C₁₋₆alkyl)aminoC₁₋₆alkyl; or
two R¹ or R² substituents adjacent to one another on the phenyl ring may independently form together a bivalent radical of formula

| | |
|---|---|
| —O—CH₂—O— | (a-1), |
| —O—CH₂—CH₂—O— | (a-2), |
| —O═CH═CH— | (a-3), |
| —O—CH₂—CH₂— | (a-4), |
| —O-CH₂—CH₂—CH₂— | (a-5), or |
| —CH═CH—CH═CH— | (a-6); |

R³ is hydrogen, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, hydroxycarbonyl, hydroxycarbonylC₁₋₆alkyl, C₁₋₆alkyloxycarbonylC₁₋₆alkyl, C₁₋₆alkylcarbonylC₁₋₆alkyl, C₁₋₆alkyloxycarbonyl, aryl, arylC₁₋₆alkyloxyC₁₋₆alkyl, mono- or di(C₁₋₆alkyl)aminoC₁₋₆alkyl;
or a radical of formula

| | |
|---|---|
| —O—R¹⁰ | (b-1), |
| —S—R¹⁰ | (b-2), |
| —NR¹¹R¹² | (b-3), |

wherein R¹⁰ is hydrogen, C₁₋₆alkyl, C₁₋₆alkylcarbonyl, aryl, arylC₁₋₆alkyl, C₁₋₆alkyloxycarbonylC₁₋₆alkyl, or a radical of formula -Alk-OR¹³ or -Alk-NR¹⁴R^{15;}
R¹¹ is hydrogen, C₁₋₆alkyl, aryl or arylC₁₋₆alkyl;
R¹² is hydrogen, C₁₋₆alkyl, aryl, hydroxy, amino, C₁₋₆alkyloxy, C₁₋₆alkylcarbonylC₁₋₆alkyl, arylC₁₋₆alkyl, C₁₋₆alkylcarbonylamino, mono- or di(C₁₋₆alkyl)amino, C₁₋₆alkylcarbonyl, aminocarbonyl, arylcarbonyl, haloC₁₋₆alkylcarbonyl, arylC₁₋₆alkylcarbonyl, C₁₋₆alkyloxycarbonyl, C₁₋₆alkyloxyC₁₋₆alkylcarbonyl, mono- or di(C₁₋₆alkyl)aminocarbonyl wherein the alkyl moiety may optionally be substituted by one or more substituents independently selected from aryl or C₁₋₃alkyloxycarbonyl, aminocarbonylcarbonyl, mono- or di(C₁₋₆alkyl)aminoC₁₋₆alkylcarbonyl, or a radical or formula -Alk-OR¹³ or -Alk-NR¹⁴R¹⁵;
wherein Alk is C₁₋₆alkanediyl;
R¹³ is hydrogen, C₁₋₆alkyl, C₁₋₆alkylcarbonyl, hydroxyC₁₋₆alkyl, aryl or arylC₁₋₆alkyl;
R¹⁴ is hydrogen, C₁₋₆alkyl, aryl or arylC₁₋₆alkyl;
R¹⁵ is hydrogen, C₁₋₆alkyl, C₁₋₆alkylcarbonyl, aryl or arylC₁₋₆alkyl;
R⁴ is a radical of formula 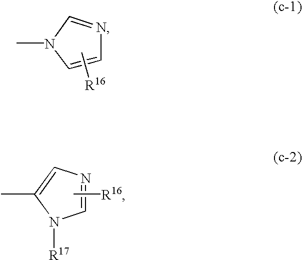
wherein R¹⁶ is hydrogen, halo, aryl, C₁₋₆alkyl, hydroxyC₁₋₆alkyl, C₁₋₆alkyloxyC₁₋₆alkyl, C₁₋₆alkyloxy, C₁₋₆alkylthio, amino, mono- or di(C₁₋₄alkyl)amino, hydroxycarbonyl, C₁₋₆alkyloxycarbonyl, C₁₋₆alkylthioC₁₋₆alkyl, C₁₋₆alkylS(O)C₁₋₆alkyl or C₁₋₆alkylS(O)₂C₁₋₆alkyl;
R¹⁶ may also be bound to one of the nitrogen atoms in the imnidazole ring of formula (c-1) or (c-2), in which case the meaning of R¹⁶ when bound to the nitrogen is limited to hydrogen, aryl, C₁₋₆alkyl, hydroxyC₁₋₆alkyl, C₁₋₆alkyloxyC₁₋₆alkyl, C₁₋₆alkyloxycarbonyl, C₁₋₆alkylS(O)C₁₋₆alkyl or C₁₋₆alkylS(O)₂C₁₋₆alkyl;
R¹⁷ is hydrogen, C₁₋₆alkyl, C₁₋₆alkyloxyC₁₋₆alkyl, arylC₁₋₆alkyl, trifluoromethyl or di(C₁₋₄alkyl)aminosulfonyl;
R⁵ is C₁₋₆alkyl, C₁₋₆alkyloxy or halo;
aryl is phenyl, naphthalenyl or phenyl substituted with 1 or more substituents each independently selected from halo, C₁₋₆alkyl, C₁₋₆alkyloxy or trifluoromethyl.

2. A combination as claimed in claim 1 wherein the farnesyl protein transferase inhibitor is a compound of formula (I) wherein X is oxygen and the dotted line represents a bond.

3. A combination as claimed in claim 1 or claim 2 wherein the farnesyl protein transferase inhibitor is a compound of formula (I) wherein R¹ is hydrogen, C₁₋₆alkyl, C₁₋₆alkyloxyC₁₋₆alkyl or mono- or di(C₁₋₆alkyl)aminoC₁₋₆alkyl and wherein R³ is hydrogen and R² is halo, C₁₋₆alkyl, C₂₋₆alkenyl, C₁₋₆alkyloxy, trihalomethoxy or hydroxyC₁₋₆alkyloxy.

4. A combination as claimed in any of the preceding claims wherein the farnesyl protein transferase inhibitor is a compound of formula (I) wherein R⁸ is hydrogen, hydroxy, haloC₁₋₆alkyl, hydroxyC₁₋₆alkyl, cyanoC₁₋₆alkyl, C₁₋₆alkyloxycarbonylC₁₋₆alkyl, imidazolyl, or a radical of formula —NR¹¹R¹² wherein R¹¹ is hydrogen or C₁₋₁₂alkyl and R¹² is hydrogen, C₁₋₆alkyl, C₁₋₆alkyloxy, C₁₋₆alkyloxyC₁₋₆alkylcarbonyl, hydroxy, or a radical of formula -Alk²-OR¹³ wherein R¹³ is hydrogen or C₁₋₆alkyl.

5. A combination as claimed in claim 1 wherein the farnesyl transferase inhibitor is selected from:
4-(3-chlorophenyl)-6-[(4-chlorophenyl)hydroxy(1-methyl-1H-imidazol-5-yl)-methyl]-1-methyl-2(1H)-quinolinone,
6-[amino(4-chlorophenyl)-1-methyl-1H-imidazol-5-ylmethyl]-4-(3-chlorophenyl)-1-methyl-2(1H)-quinolinone;
6-[(4-chlorophenyl)hydroxy(1-methyl-1H-imidazol-5-yl)methyl]-4-(3-ethoxy-phenyl)-1-methyl-2(1H)-quinolinone;
6-[(4-chlorophenyl)(1-methyl-1H-imidazol-5-yl)methyl]-4-(3-ethoxyphenyl)-1-methyl-2(1H)-quinolinone monohydrochloride.monohydrate;
6-[amino(4-chlorophenyl)(1-methyl-1H-imidazol-5-yl)methyl]-4-(3-ethoxyphenyl)-1-methyl-2(1H)-quinolinone, and
6-amino(4-chlorophenyl)(1-methyl-1H-imidazol-5-yl)methyl]-1-methyl-4-(3-propylphenyl)-2(1H)-quinolinone; a stereoisomeric form thereof or a pharmaceutically acceptable acid or base addition salts thereof.

6. A combination as claimed in claim 1 wherein the farnesyl transferase inhibitor is (+)-6-[amino(4-chlorophenyl)(1-methyl-1H-imidazol-5-yl)methyl]-4-(3-chloro-phenyl)-1-methyl-2(1H)-quinolinone; or a pharmaceutically acceptable acid addition salt thereof.

7. A combination as claimed in claim 1 wherein the farnesyl protein transferase inhibitor is a compound of formula (IX) wherein ═X¹—X²—X³ is a trivalent radical of formula (x-2), (x-3) or (x-4), >Y1—Y2 is a trivalent radical of formula (y-2), (y-3) or (y-4), r and s are 1, t is 0, R¹ is halo, preferably chloro, and most preferably 3-chloro or R¹ is C₁₋₄alkyl, preferably 3-methyl, R² is halo, preferably chloro, and most preferably 4-chloro, R³ is a radical of formula (b-1) or (b-3), R⁴ is a radical of formula (c-2), R⁶ is C₁₋₄alkyl, R⁹ is hydrogen, R¹⁰ and R¹¹ are hydrogen and R¹² is hydrogen or hydroxy.

8. A combination as claimed in claim 1 wherein the farnesyl protein transferase inhibitor is 5-(3-chlorophenyl)-α-(4-chlorophenyl)-α-(1-methyl-1H-imidazol-5-yl)tetrazolo[1,5-a]quinazoline-7-methanamine or a pharmaceutically acceptable acid addition salt thereof.

9. A combination as claimed in any of the preceding claims in which the anti-tumor nucleoside derivative is 5-fluorouracil, gemcitabine or capecitabine.

10. A combination as claimed in any of the preceding claims in the form of a pharmaceutical composition comprising an anti-tumor nucleoside derivative and a farnesyl transferase inhibitor selected from compounds of formulae (I), (II), (III), (IV), (V), (VI), (VII), (VIII) and (IX) (as defined in claim 1) together with one or more pharmaceutical carriers.

11. A combination as claimed in any of the preceding claims for use in medical therapy.

12. A combination as claimed in claim 11 for inhibiting the growth of tumor cells.

13. Use of a combination as claimed in any of claims 1 to 12 in the manufacture of a pharmaceutical composition for inhibiting the growth of tumor cells.

14. A method of inhibiting the growth of tumor cells in a human subject which comprises administering to the subject an effective amount of a combination as claimed in any of claims 1 to 12.
